(19) United States
(12) Patent Application Publication
(10) Pub. No.: US 2015/0126992 A1
(43) Pub. Date: May 7, 2015

(54) Helical DeNervation Ablation Catheter Apparatus

(71) Applicant: Jamil MOGUL, Saratoga, CA (US)

(72) Inventor: Jamil MOGUL, Saratoga, CA (US)

(73) Assignee: MOGUL ENTERPRISES, INC, Saratoga, CA (US)

(21) Appl. No.: 14/072,046

(22) Filed: Nov. 5, 2013

(51) Int. Cl. A61B 18/14

(52) U.S. Cl. CPC A61B 18/1492; A61B 2018/00434
USPC 606/34

(57) ABSTRACT

A catheter apparatus carrying RF ablation electrodes on a helically configured portion of a flexible tube that can be inserted into the femoral artery of a patient, advanced into a renal artery, and then be manipulated to properly position electrodes carried by the helical tube to contact the endoluminal surface of the artery. While instrumentally monitoring the endoluminal surface's temperature and impedance (measured with electrodes that are in an intimate contact with the surface), a low level of RF energy can be applied to selected sites on the interior (endoluminal) surface of the artery in order to ablate the renal sympathetic nerves without affecting the abdominal, pelvic, or lower-extremity nerves.

### TECHNICAL FIELD

The present invention relates generally to catheter apparatus and more specifically to a DNA catheter apparatus using radiofrequency (RF) energy to accomplish renal sympathetic denervation as a treatment for patients with drug-resistant hypertension.

### BACKGROUND

Hypertension is associated with high rates of cardiovascular disease and death. Patients with a high blood pressure are treated with a variety of antihypertensive drugs which have increasingly shown to control their blood pressure. However, various studies indicate that the control rate remains suboptimal or the blood pressure is non-respondent or resistant to the drug treatment for a subset of population.

Resistant hypertension is a failure to achieve a targeted level of blood pressure in patients who adhere to full tolerated doses of an appropriate three-drug regimen. Large clinical trials suggest that about 20% to 30% of hypertensive patients have resistant hypertension. For such patients, non-pharmacologic treatments are being tried and studied.

Various studies suggest that hyper-activation of the sympathetic nervous system is a major factor in initiating and maintaining hypertension. Direct intra-neural recordings indicate a high level of sympathetic nerve activity in the muscles of hypertensive patients who also have high levels of cardiac and renal norepinephrine (neurotransmitter) which escape neuronal uptake and local metabolism and overflow into the blood circulation.

Advances in the understanding of neural control of the kidney have demonstrated that nerves are located in the renal vessels, tubules and juxtaglomerular granular cells. At the renal arteries, these nerves are situated in the adventitial layer. Given the clear link between sympathetic renal nerves and blood pressure, a localized endovascular approach to disrupt these nerves should theoretically result in a blood pressure reduction without the unwanted side effects demonstrated in surgical sympathectomy.

With this increased understanding of the sympathetic nervous system's role in hypertension, renal sympathetic denervation has been proposed as an innovative treatment methodology targeting patients with drug-resistant hypertension. One of the renal denervation methods is to use radiofrequency (RF) energy to ablate the renal sympathetic nerves that run through the adventitia of the renal arteries in a mesh-like pattern.

### SUMMARY OF INVENTION

### Technical Problem

How to accomplish denervation of the renal sympathetic nerves which run through the adventitia of the renal arteries using radiofrequency (RF) energy without affecting the abdominal, pelvic, or lower-extremity nerves?

### Solution to the Problem

One solution to the problem is to provide a catheter apparatus carrying RF ablation electrodes on a helically configured portion of a flexible tube that can be inserted into the femoral artery of a patient, advanced into a renal artery, and then be manipulated to properly position the electrodes to contact the endoluminal surface of the artery. While instrumentally monitoring the endoluminal surface's temperature and impedance (measured with electrodes that are in an intimate contact with the surface), a low level of RF energy can be applied to selected sites on the interior (endoluminal) surface of the artery in order to ablate the renal sympathetic nerves without affecting the abdominal, pelvic, or lower-extremity nerves.

Briefly, a presently preferred embodiment of the present invention includes a catheter comprised of a flexible, soft, multi-lumened distal shaft tube carrying RF-ablation electrodes, temperature sensors and associated connecting wires on/in a helically-coiled portion (including an imbedded a pre-formed mechanical wire) of the length thereof, a less soft, flexible single or multi-lumened proximal shaft tube, a flexible sliding tube or rod, and a handle with an actuating mechanism and cable assembly/connector. In one embodiment, the handle contains a Tuohy-Borst fitting that functions as a gland seal for the sliding tube or rod. In another embodiment, the sliding tube or rod is covered with elastically-stretchable tubing that is, at one end, attached to the distal end of the helically-coiled distal shaft tube, and at the other end is attached to the distal end of the proximal shaft tube. In another embodiment, the proximal end of the sliding tube or rod is inserted into and extended out of a Tuohy-Borst fitting that is attached to a feature inside the handle in alignment with the sliding rod. In yet another embodiment, no Tuohy-Borst fitting is provided inside the handle and the sliding tube or rod is directly attached to an actuating slider within the handle mechanism.

### BRIEF DESCRIPTION OF THE DRAWING

FIG. 1 is a side elevational view showing a DNA catheter apparatus in accordance with a presently preferred embodiment of the present invention;

FIG. 2 is an enlarged view showing the distal end portion of the distal shaft tube depicted in FIG. 1 and carrying RF-ablation electrodes, temperature sensors and their wires;

FIG. 3 is an enlarged view showing the junction of the distal shaft tube/sliding rod/proximal shaft tube of FIG. 2;

FIG. 4 is a transverse cross-sectional view taken along the line 4-4 of FIG. 3;

FIG. 5 is an enlarged view of the junction of the ends of the distal shaft tube and sliding rod, and a first ablating electrode as depicted in FIG. 1;

FIG. 6 is a generalized pictorial view illustrating a segment of the distal shaft tube between the forward end of the first electrode and the intersection of the sliding rod;

FIG. 7 is a view similar to FIG. 1 depicting a longitudinal cross-section of the handle portion of the catheter apparatus with the distal end of the catheter fully extended;

FIG. 8 is a view similar to FIG. 7 but depicting a longitudinal cross-section of the handle portion of the catheter apparatus with the distal end of the catheter retracted to expand the electrodes into an operative disposition;

FIG. 9 is an enlarged view of the distal end portion of the retracted catheter showing the expanded electrodes depicted in FIG. 8;

FIG. 10 is a side elevational/cross-sectional view showing an alternative embodiment of a catheter apparatus in accordance with the present invention with the distal end fully extended;

FIG. 11 is a view similar to FIG. 10 but depicting the distal end retracted to expand the electrodes into an operative disposition;

FIG. 12 is an enlarged view showing a distal end portion of the distal shaft tube which carries the RF-ablation electrodes and temperature sensors and their wires;

FIG. 13 is a tranverse cross-sectional view taken along the line 13-13 of FIG. 12;

FIG. 14 is a generalized pictorial view illustrating a segment of the distal shaft tube at its intersection with the sliding rod;

FIG. 15 is an enlarged view of the junction of the ends of the distal shaft tube and sliding rod, and a first ablating electrode as depicted in FIG. 10; and

FIG. 16 is a generalized pictorial view illustrating the segment of the distal shaft tube between the forward end of the first electrode and its intersection with the sliding rod;

### DESCRIPTION OF THE EMBODIMENTS

The ensuing description provides preferred exemplary embodiment(s) only, and is not intended to limit the scope, applicability, or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as set forth in the appended claims.

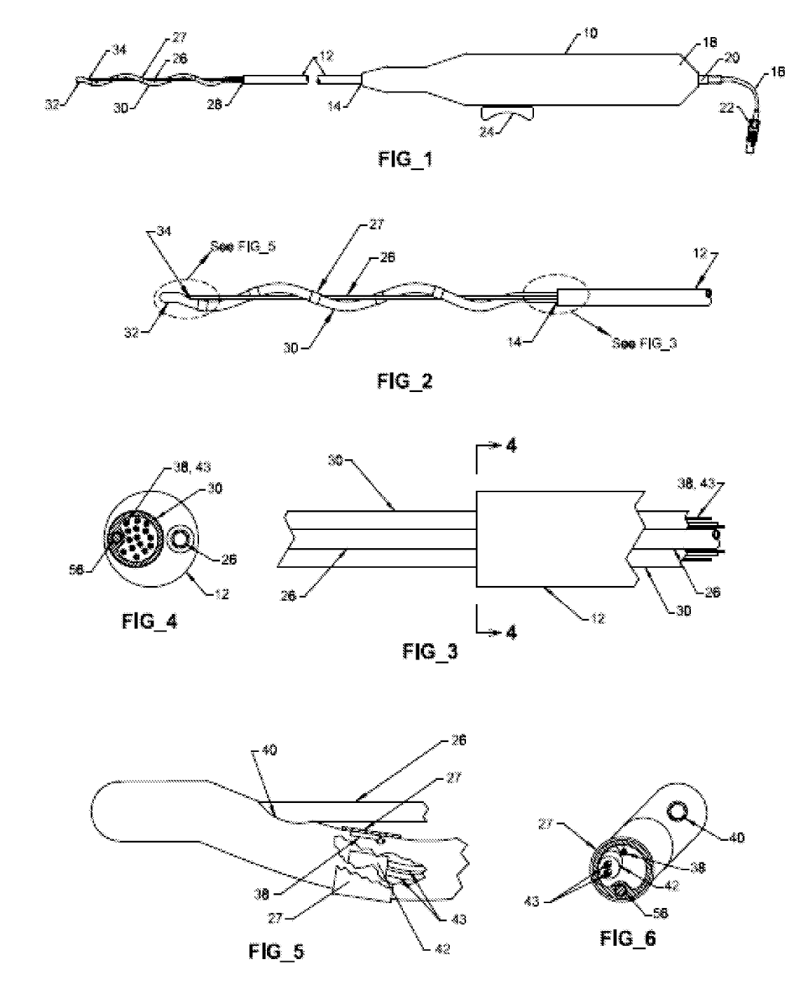

Referring now to FIG. 1 of the Drawing, a DNA catheter apparatus in accordance with a first embodiment of the present invention is shown to include a handle 10 having a polymeric tubular proximal shaft 12 attached to and extending from one end 14, and a cable assembly 16 extending from the other end 18 through a polymeric strain relief fitting 20 and terminating in a suitable electrical connector 22. The handle 10 also serves as a housing for a relatively simple actuating mechanism, to be described below, which links a plastic or metallic slider 24 disposed in a slideable relationship with a slot (not shown) formed along a bottom portion of the length of the handle. The slider is operatively connected to a flexible tube or rod 26 made of a plastic or metallic material and extending out of the handle and through the flexible proximal shaft 12, and is internally in a forced contact with a frictional pad (not shown) mounted on a wall of the handle so as to auto-lock the slider at a desired position.

Also extending out of the distal end 28 of shaft 12 is a soft, flexible, multi-lumened distal shaft tube 30, typically made of a suitable polymeric material and carrying a plurality of metallic electrodes 27, preferably made of platinum/iridium. Although depicted as cylindrical metallic rings, the electrodes 27 can alternatively be conductive strips of material wrapped about tube 30 like a ribbon, or lengths of wire-like conductive material wrapped or coiled about tube 30 in helical, toroidal or semi-toroidal fashion. They could also be shaped as longitudinally split or semi-cylindrical bands, circular dots, meshed pads, rectangular strips, or circumferentially-printed metal.

Tube 30, internally containing a helically-coiled mechanical wire (not shown), is wrapped in a helical fashion about the rod 26 and is affixed at its distal extremity 32 to the distal end 34 of rod 26. As depicted, the rod 26 is disposed in its maximally extended disposition, and the tubing 30 is disposed in its maximally stretched disposition and thus wrapped relatively tightly about rod 26, as is perhaps better shown in FIG. 2, to facilitate insertion into a patient's artery.

As best depicted in FIGS. 2 and 5, the exterior of the helically-coiled distal shaft tube 30 is provided with multiple, cylindrically configured electrodes 27 that are spaced at predetermined intervals along the length of the helically coiled portion of the tube. The interior of the tube 30 contains a helically-coiled mechanical wire 56 (see FIGS. 4, 6), and multiple polymer coated (insulated) electrical wires 38, 43 (see FIGS. 3 and 4), some of which extend through an opening (FIG. 5) in the tube 30 under a corresponding electrode 27 and are electrically connected to the electrode. The helically-coiled wire 56 resides within the tube 30 and extends internally of and along the length of the tube 30. Wire 56 is preferably made of a spring stainless steel or nitinol (nickel titanium alloy), and at least a portion thereof is preferably pre-formed into a helical shape before it is inserted into the distal shaft tube 30. Alternatively, the wire 56 could be made of any suitable flexible plastic material having appropriate “spring” characteristics.

At each electrode 27, a suitable temperature sensor 42 (FIGS. 5, 6) is connected to electrical wires 43 and disposed within the tube 30 at the same location as the electrode. The insulated (with a polymeric or other suitable coating) electrical wires 43 leading to the sensors 42 are bundled together with the similarly insulated wires 38 connected to the electrodes 27 and extend internally of and along the length of the distal shaft tube 30 and back through the proximal shaft 12, handle 10 and fitting 20, ultimately terminating at the cable connector 22.

Turning now to FIG. 7 which includes the showing of a longitudinal cross section of the handle 10, the internal mechanical details of the handle, and the slider assembly. Handle 10 is preferably made of a 2-part, injection molded or otherwise formed plastic assembly, typically split down a longitudinal center-line thereof, for housing a linear actuator such as the slider 24 and associated means for guiding and coupling the slider to the coil expanding/retracting rod 26. Handle 10 also provides a pathway through which the cable 16 may be passed for extension into the proximal end 44 of the proximal shaft tube 12. A Tuohy-Borst fitting 45, typically made of plastic and rubber materials, is also contained within handle 10 and is disposed near the end 44 of tube 12 to function as a gland seal for the sliding tube or rod 26.

The essentially mirror-imaged parts forming the handle/housing 10 are either fastened together with suitable screw fasteners or the like (not shown), or are configured to snap-lock together. Each of the two handle parts, only one of which is depicted in FIG. 7, includes semi-cylindrical recesses 46 and 48 that, when the two parts are mated together, form cylindrical ports, at each end of the handle, through which the proximal shaft tube 12, and the fitting 20 are respectively fastened. The mated handle parts also include elongated notches formed in their lower edges that, when the two parts are mated together, form a slot 50 through which a necked down portion 51 of the slider 24 extends. The slider portion 51 is slidingly captured within the slot 50 and held in a forced contact relationship with a frictional pad 53 mounted inside the handle. The pad 53 is typically made of a polymeric or rubber-like material and acts as an auto-brake or auto-lock for the slider 10.

Grooves or channel forming ridges 52 are also formed on the interior walls of the handle parts and combine to form a track along which shoulders 54 formed on the slider engage and slide as the slider is moved from one end of the slot 50 to the other.

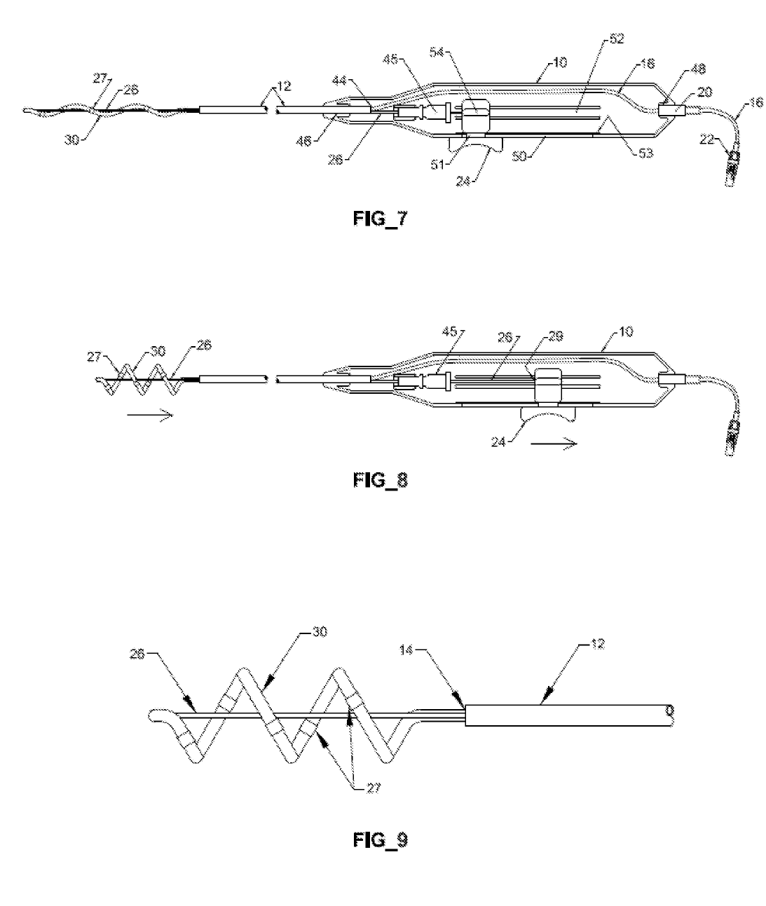

In use, with the distal end of the catheter inserted into position within an artery to be treated, the operating technician or physician will move the slider 24 rightwardly, as illustrated in FIG. 8, to withdraw the rod 26 thereby pulling the distal end of the catheter toward the handle, collapsing the helix and thereby reducing the pitch and increasing the diameter of the coiled tube 30. and thus changing the longitudinal and radial loci of the several electrodes 27 as illustrated in FIGS. 8 and 9.

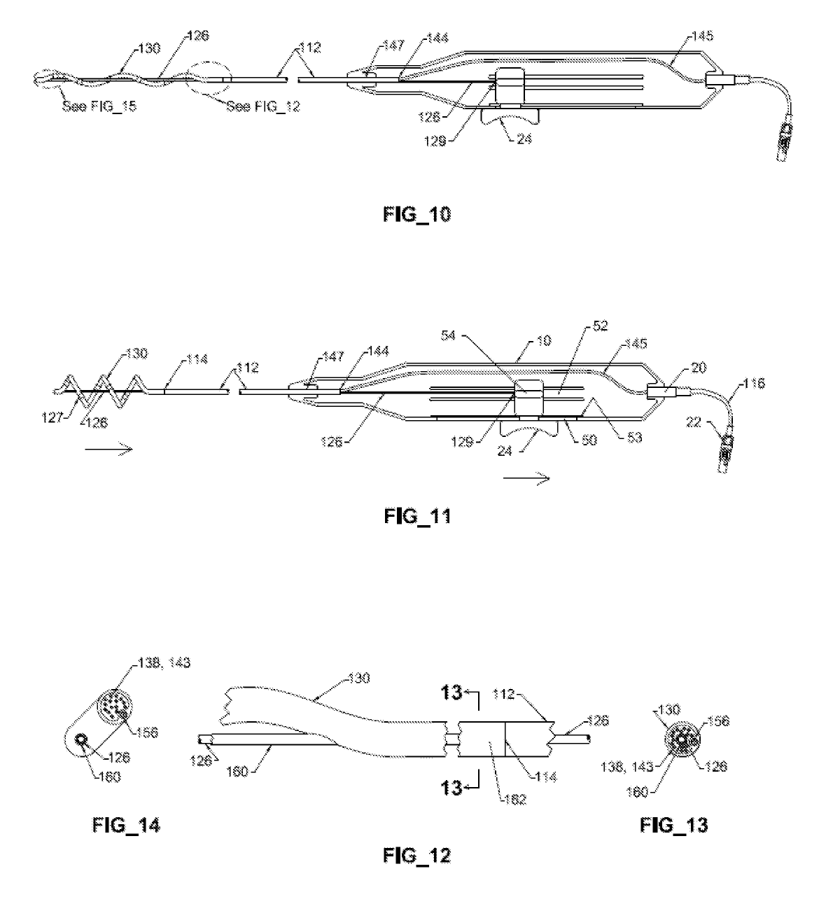

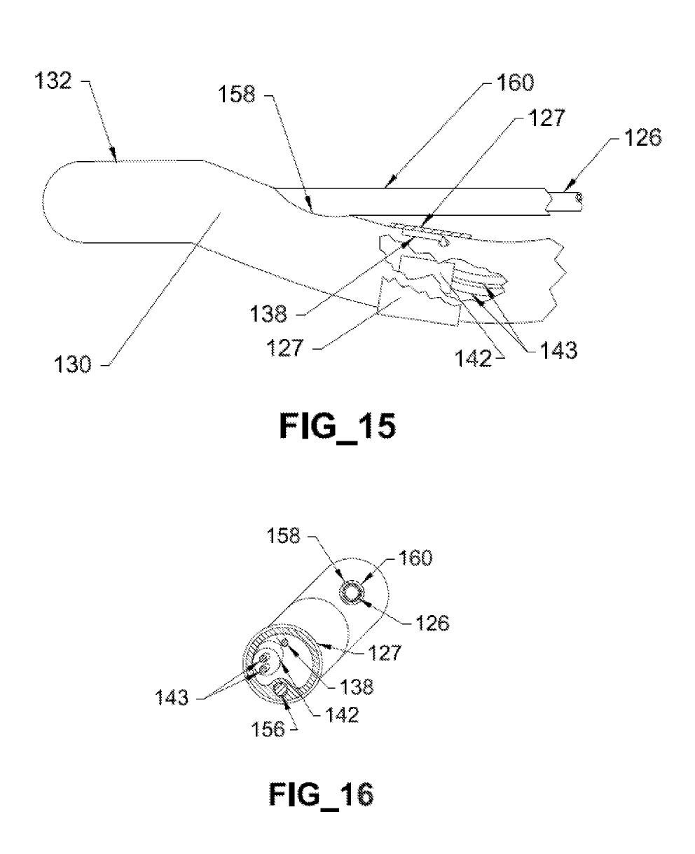

In an alternative embodiment illustrated in FIGS. 10-16, instead of using a Tuohy-Borst fitting to provide a seal for the sliding tube or rod 126, the tube or rod 126 is covered with an elastically-stretchable tubing 160 (see FIGS. 15-16) that is, at one end 158, attached to the distal end 132 of the helically-coiled distal shaft tubing 130, and at the other end 162, attached to the distal end 114 of the proximal shaft tubing 112.

The electrical wires 138, 143 and sliding tube or rod 126 pass through the proximal shaft tubing 112 and extend out of the proximal end 144 thereof. At its proximal end, the proximal shaft tubing 112 is attached to the handle at 147, and the bundled electrical wires 138, 143 are encapsulated within tubing 145 which extends along the length of the handle 110, and then spliced to the conductors of the cable assembly 116 and associated connector 22 that is attached to the back end of the handle 10.

Functionally, in the first above described embodiment, the helically-coiled distal shaft tubing is designed to be extended in its rest or relaxed state, and is then compressed, as needed to deploy the several contacts into engagement with an endoluminal surface to be treated, by moving the slider rearwardly within the handle. But in the second embodiment, the helically-coiled-distal shaft tubing is designed to be compressed when in its rest or relaxed state, and to be extendable into an operating disposition by moving the slider forward. In each case, the extended state is for accommodating insertion of the catheter into an artery to be treated, and the retracting action is for adjusting, expanding and fitting the helix of the helically-coiled distal shaft and its carried electrodes within the artery.

Clinically, the helically-coiled ablation catheter, in an extended state, will be inserted into a femoral artery and advanced into a renal or other artery to be treated or ablated. Then the catheter will be connected to an RF generator via the cable assembly/connector. The catheter's RF-ablation electrode(s) will then be deployed by movement of the slider and made to contact the endoluminal surface of the artery as needed. While monitoring the temperature and impedance of the engaged endoluminal surface, a low level of RF energy will be applied via the several electrodes to selected sites on the endoluminal, or inner arterial, surface of the artery in order to ablate the renal sympathetic nerves contained therein without affecting the abdominal, pelvic, or lower-extremity nerves.

The clinical procedure is normally performed on both right and left renal arteries, with four to six selected sites being ablated in a longitudinal and rotational configuration in 2-minute RF-applications at each site, in order to cover the full circumference of the arteries.

Although the actuator assembly used to deploy the catheter electrodes has been depicted as including a slider mechanism, it is contemplated that alternative actuating mechanisms could also be used. For example, other means for enabling extension and retraction of the rod or tube 26 might include or be embodied as a user engageable, pivotable lever and fork engaging a fixture at the proximal end of the rod; a user engageable pivotable lever-driven gear and rack attached to the end of the rod; a thumb wheel driven screw attached to the end of the rod; a thumb driven rotatable pinion and rack attached to the end of the rod; a simple hydraulic or pneumatic actuator of some type; a ball screw actuator, or a low voltage electrical motor driven version of any of these.

Although the present invention has been described above in terms of several alternative embodiments, and various modifications and applications have been suggested, it is anticipated that after reading the foregoing disclosure, numerous other embodiments and applications of the present invention will become apparent to those skilled art. For example, the disclosed actuating mechanism could be embodied separate from the catheter handle by extending the sliding rod or tube through the handle to a separate actuator. It is therefore intended that this disclosure be considered as exemplary rather than limiting, and that the following claims be interpreted as covering all alternatives, modifications and embodiments as fall within the true spirit and scope of the invention.

### APPLICATION CALL-OUT NUMBER LIST

handle 10

proximal shaft tube 12

one handle end 14

cable assembly 16

another handle end 18

strain relief fitting 20

connector 22

linear actuator/slider 24

flexible tube or rod 26

electrodes 27

distal end 28

proximal end 29

distal shaft tube 30

distal extremity 32

distal end 34

electrical wires 38

electrical wires 43

radial opening 40

temperature sensor 42

proximal end 44

Tuohy-Borst fitting 45

recesses 46 and 48

slot 50

necked down portion 51

grooves or channel forming ridges 52

frictional pad 53

shoulders 54

helically-coiled mechanical wire 56

proximal shaft tubing 112

distal end 114

cable assembly 116

sliding tube or rod 126

helically-coiled distal shaft tube portion 130

distal end 132

electrical wires 138, 143

proximal end 144

Attachment 147

helically-coiled mechanical wire 156

one end 158

elastically-stretchable tubing 160

other end 162

1. Catheter apparatus for applying RF energy to the endoluminal surfaces of a patient's arteries to ablate certain nerves contained therein, comprising:
a handle having a distal end and a proximal end, and having a linear actuator means associated therewith;
a proximal shaft tube affixed to the distal end of the handle;
a flexible tube or rod extending through the proximal shaft tube and having its proximal end coupled to the linear actuator means;
a flexible distal shaft tube having its proximal end affixed to the distal end of the proximal shaft tube, its distal end connected to the distal end of the flexible tube or rod, and having a helically configured portion of its length helically wrapped about a distal portion of the flexible tube or rod;
a plurality of electrodes and temperature sensors carried by and disposed at predetermined intervals along the helically configured portion of the distal shaft tube; and
a bundle of electrical wires extending through and along at least a portion of the distal shaft tube and the proximal shaft tube, various ones of the wires having their respective distal ends connected to the electrodes or the temperature sensors, and their proximal ends coupled to electrical connector means for connection to electrical power and measurement means;
whereby selective actuation of the linear actuator means causes extension or retraction of the flexible tube or rod which in turn causes the helical pitch and radius of the helically configured portion of the distal shaft tube to be changed to variously position said electrodes within an artery into which said catheter is extended.

2. Catheter apparatus as recited in claim 1 wherein the linear actuator means is contained within the handle and includes a user engagable mechanism extending through an opening in the handle.

3. Catheter apparatus as recited in claim 2 wherein the user engagable mechanism is a slider affixed to the proximal end of the flexible tube or rod, the slider extending out of the handle through a slot formed therein and being movable there along to extend or retract the flexible tube or rod.

4. Catheter apparatus as recited in claim 3 wherein the bundle of electrical wires extends into the handle at the distal end and exits at the proximal end.

5. Catheter apparatus as recited in claim 1 wherein the electrodes are in the form of metal bands circumscribing segments of the helically configured portion of the distal shaft tube.

6. Catheter apparatus as recited in claim 5 wherein the sensors are disposed within the helically configured portion of the distal shaft tube and each sensor is circumscribed by a corresponding metal band such that the temperature of the distal shaft tube at the corresponding electrode may be monitored.

7. Catheter apparatus as recited in claim 1 wherein the sensors are disposed within the helically configured portion of the distal shaft tube and each sensor is circumscribed by a corresponding metal band such that the temperature of the distal shaft tube at the corresponding metal band may be monitored.

8. Catheter apparatus as recited in claim 2 and further comprising a Tuohy-Borst fitting disposed within the handle and between the proximal end of the proximal shaft tube and the linear actuator, the fitting functioning as a gland seal for the sliding tube or rod which extends therethrough.

9. Catheter apparatus as recited in claim 1 and further comprising:
a pre-formed helically coiled resilient wire embedded in and extending along at least the selectively configurable portion of the distal portion of the flexible distal shaft tube.

10. A catheter device for applying RF energy to the endoluminal surfaces of an artery or the like, comprising:
a handle having a distal end and a proximal end, and having a linear actuator means disposed therewithin;
a length of flexible distal shaft tube having a distal end and a proximal end, the proximal end thereof being coupled to the distal end of the handle, and having a portion of its length near its distal end being helically configured;
a flexible rod slideably extending through and along a substantial portion of the length of the distal shaft tube not including the helically configured portion thereof, the flexible rod having its proximal end coupled to the linear actuator means, and a distal end portion thereof extending out of a sidewall of the distal shaft tube and along the axis of the helically configured portion thereof, the distal end of the rod being affixed to the distal end of the distal shaft tube;
a plurality of electrodes and temperature sensors carried by and disposed at predetermined intervals along the helically configured portion of the distal shaft tube; and
a bundle of electrical wires extending through and along at least a portion of the distal shaft tube, various ones of the wires having their distal ends variously connected to the electrodes or the temperature sensors, and their proximal ends coupled to electrical connector means for connection to RF power providing and temperature measurement means;
whereby selective actuation of the linear actuator means causes linear extension/retraction of the flexible rod which in turn causes the helical pitch and radius of the helically configured portion of the distal shaft tube to be changed to variously position the electrodes within an artery into which the catheter is inserted.

11. A catheter device as recited in claim 10 wherein the linear actuator means contained within the handle includes a user engagable mechanism extending through an opening in the handle.

12. A catheter device. as recited in claim 11 wherein the user engagable mechanism is a slider affixed to the proximal end of the flexible rod, the slider extending out of the handle through a slot formed therein and being movable there along to extend or retract the flexible rod.

13. A catheter device as recited in claim 10 wherein the electrodes are in the form of metal bands circumscribing segments of the helically configured portion of the distal shaft tube.

14. A catheter device as recited in claim 13 wherein the sensors are disposed within the helically configured portion of the distal shaft tube and each sensor is circumscribed by a corresponding metal band such that the temperature of the distal shaft tube at the corresponding electrode may be monitored.

15. A catheter device as recited in claim 14 wherein the sensors are disposed within the helically configured portion of the distal shaft tube and each sensor is circumscribed by a corresponding electrode forming conductor such that the temperature of the distal shaft tube at the corresponding electrode may be monitored.

16. A catheter device as recited in claim 10 and further comprising a Tuohy-Borst fitting disposed within the handle and between the proximal end of the distal shaft tube and the linear actuator, the fitting functioning as a gland seal for the sliding rod which extends therethrough.

17. A catheter device as recited in claim 10 and further comprising:
a resilient wire embedded in and extending along at least a portion of the length of the distal shaft tube, the resilient wire having a helically configured portion thereof being embedded in the helically configured portion of the distal shaft tube.

18. A catheter device as recited in claim 10 wherein the proximal end of the distal shaft tube is coupled to the distal end of the handle by a proximal shaft tube.

19. A catheter device as recited in claim 18 wherein the flexible rod is covered with an elastically-stretchable tubing having one end thereof sealingly attached to the distal end of the distal shaft tube and the other end thereof sealingly attached to the distal end of the proximal shaft tube to provide a seal for the flexible rod.

20. A catheter device as recited in claim 10 and further comprising:
a length of resilient plastic rod having a pre-formed helically coiled portion thereof embedded in and extending along the helically configured portion of the flexible distal shaft tube.
